(19) United States
(12) Patent Application Publication
(10) Pub. No.: US 2017/0218409 A1
(43) Pub. Date: Aug. 3, 2017

(54) Production of Acrylate in Cells

(71) Applicant: President and Fellows of Harvard College, Cambridge, MA (US)

(72) Inventors: Jameson K. Rogers, Boston, MA (US); George M. Church, Brookline, MA (US)

(21) Appl. No.: 15/421,495

(22) Filed: Feb. 1, 2017

Related U.S. Application Data
(60) Provisional application No. 62/289,541, filed on Feb. 1, 2016.

(51) Int. Cl. C12P 7/62; C12N 15/52

(52) U.S. Cl. CPC C12P 7/62; C12N 15/52; C12Y 103/01084; C12Y 602/00

(57) ABSTRACT

A method of producing acrylate in vivo in a genetically modified bacteria cell is provided including providing a cell with a first one or more nucleic acids encoding a pathway resulting in the production of 3-hydroxypropionate, providing the cell with a second one or more nucleic acids encoding a truncated pcs enzyme lacking a domain converting acrylyl-CoA to propionyl-CoA, providing the cell with a third one or more nucleic acids encoding acrylyl-CoA hydrolase, wherein the cell expresses the first, second and third one or more nucleic acids and produces acrylate.

### RELATED APPLICATION DATA

This application claims priority to U.S. Provisional Patent Application No. 62/289,541, filed on Feb. 1, 2016 and is hereby incorporated herein by reference in its entirety for all purposes.

### STATEMENT OF GOVERNMENT INTERESTS

This invention was made with government support under DE-FG02-02ER63445 awarded by the U.S. Department of Energy. The government has certain rights in the invention.

### FIELD

The present invention relates in general to genetically modified bacteria that produce acrylate.

### BACKGROUND

Acrylate is an important industrial chemical that is primarily produced from petroleum feedstocks. Previous work has demonstrated production of the acrylate precursor 3-hydroxypropionate (3HP) from glucose using an engineered strain of E. coli. See Rathnasingh et al. (2012). “Production of 3-hydroxypropionic acid via malonyl-CoA pathway using recombinant Escherichia coli strains.” Journal of Biotechnology 157:633-640. Briefly, malonyl-CoA (which E. coli produces from glucose naturally) is converted to 3HP by the heterologous enzyme malonyl-CoA reductase. Other methods exist to produce 3HP from sugar, sunlight or other biomass feedstocks. See Kumar et al. (2013). “Recent advances in biological production of 3-hydroxypropionic acid.” Biotechnology Advances 31:945-961. Methods are known to convert 3HP to acrylate utilizing a non-enzymatic chemical reaction to dehydrate 3HP which is isolated from the cell and using high temperatures and an inorganic catalyst. See Straathof et al. (2005). “Feasibility of acrylic acid production by fermentation.” Appl Microbiol Biotechnol 67:727-734.

### SUMMARY

Embodiments of the present disclosure are directed to methods of genetically modifying bacteria cells, or microbes, to produce acrylate. It is to be understood that at the pH within the bacteria cell, the cell produces acrylate, but should the acrylate be exposed to conditions causing its protonation, acrylic acid may be produced. Embodiments of the present disclosure are directed to genetically modified bacteria cells, or microbes, that include an acrylate biosynthesis pathway from glucose to acrylate. The disclosure provides that a bacteria cell is genetically modified to include one or more nucleic acids encoding enzymes that convert glucose to 3-hydroxypropionate and then convert 3-hydroxypropionate to acrylate within the genetically modified bacteria cell in vivo. The acrylate is produced directly within the cell. The acrylate is then harvested from the bacteria cell. The disclosure provides that culture conditions are provided within which a genetically modified bacteria cell that produces acrylate as described herein is cultured to produce acrylate.

The disclosure provides that a bacteria cell is genetically modified to produce acrylate using an enzymatic route that converts glucose, or other feedstock, to acrylate in a biological system in vivo. Enzymes or nucleic acids encoding such enzymes may be exogenous or foreign. Exogenous or foreign enzymes or nucleic acids are non-native to the cell. The disclosure provides that a bacterial cell is genetically modified to produce 3-hydroxypropionate, such as by including exogenous DNA encoding genes to produce 3-hydroxypropionate. The disclosure provides that a bacteria cell is genetically modified to include one or more nucleic acids encoding the biosynthetic pathway as shown in FIG. 1. See Rathnasingh et al. (2012). “Production of 3-hydroxypropionic acid via malonyl-CoA pathway using recombinant Escherichia coli strains.” Journal of Biotechnology 157:633-640 hereby incorporated by reference in its entirety. The cell accordingly produces 3HP.

The disclosure provides that the bacteria cell is genetically modified in a manner to include exogenous or foreign enzymes such that 3HP is converted to 3-hydroxy-propionyl-CoA which is then converted to acrylyl-CoA by a truncated version of the heterologously expressed propionyl-coenzyme A synthase (PCS) from Chloroflexus aurantiacus. See Alber and Fuchs. (2002). “Propionyl-Coenzyme A Synthase from Chloroflexus aurantiacus, a Key Enzyme of the 3-Hydroxypropionate Cycle for Autotrophic CO2 Fixation.” J. Biological Chemistry 277:12137-12143 hereby incorporated by reference in its entirety. The disclosure provides that a bacteria cell is genetically modified to include one or more nucleic acids encoding the truncated version of PCS (truncPCS). Native PCS catalyzes three consecutive reactions which convert 3HP to 3HP-CoA, 3HP-CoA to acrylyl-CoA, and acrylyl-CoA to propanoyl-CoA. However, the truncPCS lacks the third catalytic domain of the enzyme and the native reaction stops at the acrylyl-CoA intermediate. Accordingly, the bacteria cell is genetically modified with one or more nucleic acids encoding the pathway to produce acrylyl-CoA but not propanoyl-CoA.

The disclosure provides that the bacteria cell is genetically modified to convert acrylyl-CoA to acrylate within the cell. The disclosure provides that the bacteria cell is genetically modified to include one or more nucleic acids encoding the heterologous enzyme, acrylyl-CoA hydrolase (ACH) from Acinetobacter sp. ADP1. See Valle et al. “Direct biocatalytic production of acrylic acid and other carboxylic acid compounds.” WO2013044076 A1 hereby incorporated by reference in its entirety. The ACH enzyme converts acrylyl-CoA to acrylate within the cell.

The disclosure provides that a bacterial cell is genetically modified to produce 3-hydroxypropionate, a truncated version of PCS (truncPCS) and acrylyl-CoA hydrolase. The disclosure provides that the cell is genetically modified to produce acrylate in vivo.

The disclosure provides that a bacteria cell, such as E. coli, is genetically modified or transformed with one or more plasmids facilitating expression of malonyl CoA reductase (“MCR”), truncPCS and ACH. The genetically modified bacteria cell produces acrylate from a carbon source which the bacteria can metabolize.

The disclosure provides that fatty acid synthesis by the genetically modified bacteria cell is inhibited. The disclosure provides that the genetically modified bacteria cell is cultured in the presence of a fatty acid biosynthesis inhibitor. An exemplary fatty acid biosynthesis inhibitor is cerulenin. Other exemplary fatty acid biosynthesis inhibitors commercially available from, for example, Sigma-Aldrich, and include apigenin, C75, catechin hydrate, epigallocatechin gallate, GSK837149A, irgasan, kaempferol, luteolin and osthole. Other exemplary fatty acid biosynthesis inhibitors can readily be identified by those of skill in the art based on the present disclosure. The disclosure provides that presence of cerulenin inhibits fatty acid biosynthesis and increases levels of malonly-CoA which increases carbon flux through the acrylate biosynthetic pathway described herein and which is included in the genetically modified bacteria.

The disclosure provides that the genome of a microorganism is genetically modified to include one or more DNA sequences, such as a synthetic or foreign DNA sequence, encoding one or more enzymatic pathways which utilize a carbon source to produce 3HP and then convert 3HP to acrylyl-CoA which is then converted to acrylate within the cell. As DNA is included or inserted into the genome of the microorganism, the resulting microorganism may be referred to as a recombinant or genetically modified microorganism.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts an exemplary enzymatic pathway producing 3HP from biomass.

FIG. 2A depicts a biosynthetic pathway converting 3HP to acrylate using truncPCS and ACH. FIG. 2B depicts biosensor data demonstrating acrylate production from a genetically modified bacteria including the enzymatic pathway of FIG. 2A. FIG. 2C depicts biosensor data demonstrating that acrylate is not produced in the absence of truncPCS.

FIG. 2D depicts biosensor data demonstrating that cells without the acuR-based acrylate sensor helper plasmids do not produce fluorescence in the presence of 3HP. The fluorescent response to the addition of acrylate indicates that the biosensor is functional, but insulated against activation by 5 mM 3HP without the enzymes necessary to convert 3HP to acrylate.

FIG. 3A depicts a biosynthetic pathway for 3HP using glucose or other carbon feedstock. The 3HP can then be converted to acrylate using the biosynthetic pathway of FIG. 2A. FIG. 3B depicts use of the acrylate sensor acuR to demonstrate acrylate and thus, 3HP production. FIG. 3B depicts use of the acrylate sensor acuR to demonstrate acrylate and, thus, 3HP production.

FIG. 4A depicts data showing optimized acrylate production in the presence of cerulenin and isopropyl β-D-1-thiogalactopyranoside (“IPTG”). FIG. 4B depicts data showing optimized acrylate production in the presence of cerulenin and IPTG. FIG. 4C depicts data showing optimized acrylate production in the presence of cerulenin and IPTG.

FIG. 5A depicts data showing 3HP production. FIG. 5B depicts data showing acrylate production.

### DETAILED DESCRIPTION

The disclosure provides the genetic modification of a cell, such as a bacterial cell, a yeast cell, or a fungal cell, to produce acrylate in vivo. The cell may be genetically modified to include one or more pathways, such as exogenous pathways or pathways foreign to the cell, which function to produce acrylate. The one or more pathways include one or more genes which when expressed allow the cell to produce acrylate from a desired starting point, typically a low-cost feedstock such as glucose or biomass.

The disclosure provides a method of producing acrylate in vivo in a genetically modified bacteria cell is provided including providing a cell with a first one or more nucleic acids encoding a pathway resulting in the production of 3-hydroxypropionate, providing the cell with a second one or more nucleic acids encoding a truncated pcs enzyme lacking a domain converting acrylyl-CoA to propionyl-CoA, providing the cell with a third one or more nucleic acids encoding acrylyl-CoA hydrolase, and wherein the cell expresses the first, second and third one or more nucleic acids and produces acrylate.

The disclosure provides that the cell produces 3HP which is converted by the truncated pcs enzyme to acrylyl-CoA which is converted to acrylate by the acrylyl-CoA hydrolase. The disclosure provides that the acrylate is recovered from the cell. The disclosure provides that fatty acid biosynthesis within the cell is inhibited. The disclosure provides that fatty acid biosynthesis within the cell is inhibited by exposing the cell to a fatty acid inhibitor. The disclosure provides that fatty acid biosynthesis within the cell is inhibited by exposing the cell to cerulenin. The disclosure provides that fatty acid biosynthesis within the cell is inhibited thereby increasing malonyl-CoA for processing by malonyl-CoA reductase.

The disclosure provides that the genetically modified bacteria cell is grown into a population of genetically modified bacteria cells and the acrylate is recovered.

The disclosure provides that a method of genetically modifying a bacteria cell is provided including providing a cell with a first one or more foreign or exogenous nucleic acids encoding a pathway resulting in the production of 3-hydroxypropionate, providing the cell with a second one or more foreign or exogenous nucleic acids encoding a truncated pcs enzyme lacking a domain converting acrylyl-CoA to propionyl-CoA, and providing the cell with a third one or more foreign or exogenous nucleic acids encoding acrylyl-CoA hydrolase.

The disclosure provides that a genetically modifying a bacteria cell is provided that includes a first one or more foreign or exogenous nucleic acids encoding a pathway resulting in the production of 3-hydroxypropionate, a second one or more foreign or exogenous nucleic acids encoding a truncated pcs enzyme lacking a domain converting acrylyl-CoA to propionyl-CoA, and a third one or more foreign or exogenous nucleic acids encoding acrylyl-CoA hydrolase.

Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety.

Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

Microorganisms may be genetically modified to delete genes or incorporate genes by methods known to those of skill in the art. Vectors and plasmids useful for transformation of a variety of host cells are common and commercially available from companies such as Invitrogen Corp. (Carlsbad, Calif.), Stratagene (La Jolla, Calif.), New England Biolabs, Inc. (Beverly, Mass.) and Addgene (Cambridge, Mass.).

Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IP_{L}, IP_{R}, T7, tac, and trc (useful for expression in Escherichia coli and Pseudomonas); the amy, apr, npr promoters and various phage promoters useful for expression in Bacillus subtilis, and Bacillus licheniformis; nisA (useful for expression in Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). Termination control regions may also be derived from various genes native to the preferred hosts.

Certain vectors are capable of replicating in a broad range of host bacteria and can be transferred by conjugation. The complete and annotated sequence of pRK404 and three related vectors-pRK437, pRK442, and pRK442(H) are available. These derivatives have proven to be valuable tools for genetic manipulation in Gram-negative bacteria (Scott et al., Plasmid 50(1):74-79 (2003)). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

Chromosomal gene replacement tools are also widely available. For example, a thermosensitive variant of the broad-host-range replicon pWV101 has been modified to construct a plasmid pVE6002 which can be used to create gene replacement in a range of Gram-positive bacteria (Maguin et al., J. Bacteriol. 174(17):5633-5638 (1992)). Additionally, in vitro transposomes are available to create random mutations in a variety of genomes from commercial sources such as EPICENTRE® (Madison, Wis.).

Vectors useful for the transformation of E. coli are common and commercially available. For example, the desired genes may be isolated from various sources, cloned onto a modified pUC19 vector and transformed into E. coli host cells. Alternatively, the genes encoding a desired biosynthetic pathway may be divided into multiple operons, cloned onto expression vectors, and transformed into various E. coli strains.

The Lactobacillus genus belongs to the Lactobacillales family and many plasmids and vectors used in the transformation of Bacillus subtilis and Streptococcus may be used for Lactobacillus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (van Kranenburg R, Golic N, Bongers R, Leer R J, de Vos W M, Siezen R J, Kleerebezem M. Appl. Environ. Microbiol. 2005 March; 71(3): 1223-1230), which may be used for transformation.

Initiation control regions or promoters, which are useful to drive expression of the relevant pathway coding regions in the desired Lactobacillus host cell, may be obtained from Lactobacillus or other lactic acid bacteria, or other Gram-positive organisms. A non-limiting example is the nisA promoter from Lactococcus. Termination control regions may also be derived from various genes native to the preferred hosts or related bacteria.

The various genes for a desired biosynthetic or other desired pathway may be assembled into any suitable vector or vectors, such as those described above. A single vector need not include all of the genetic material encoding a complete pathway. One or more or a plurality of vectors may be used in any aspect of genetically modifying a cell as described herein. The codons can be optimized for expression based on the codon index deduced from the genome sequences of the host strain, such as for Lactobacillus plantarum or Lactobacillus arizonensis. The plasmids may be introduced into the host cell using methods known in the art, such as electroporation, as described in any one of the following references: Cruz-Rodz et al. (Molecular Genetics and Genomics 224:1252-154 (1990)), Bringel and Hubert (Appl. Microbiol. Biotechnol. 33: 664-670 (1990)), and Teresa Alegre, Rodriguez and Mesas (FEMS Microbiology Letters 241:73-77 (2004)). Plasmids can also be introduced to Lactobacillus plantatrum by conjugation (Shrago, Chassy and Dobrogosz Appl. Environ. Micro. 52: 574-576 (1986)). The desired biosynthetic pathway genes can also be integrated into the chromosome of Lactobacillus using integration vectors (Hols et al. Appl. Environ. Micro. 60:1401-1403 (1990); Jang et al. Micro. Lett. 24:191-195 (2003)).

Microorganisms which may serve as host cells and which may be genetically modified to produce recombinant microorganisms as described herein may include one or members of the genera Clostridium, Escherichia, Rhodococcus, Pseudomonas, Bacillus, Lactobacillus Saccharomyces, and Enterococcus. Particularly suitable microorganisms include Escherichia coli, Bacillus subtilis, and Saccharomyces cerevisiae.

Exemplary genus and species of bacteria cells include Acetobacter aurantius, Acinetobacter bitumen, Actinomyces israelii, Agrobacterium radiobacter, Agrobacterium tumefaciens, Anaplasma Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaninogenicus (also referred to as Prevotella melaninogenica), Bartonella, Bartonella henselae, Bartonella quintana, Bordetella, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cep acia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila Chlamydophila pneumoniae (also known as Chlamydia pneumoniae) Chlamydophila psittaci (also known as Chlamydia psittaci), Clostridium, Clostridium botulinum, Clostridium difficile, Clostridium perfringens (also known as Clostridium welchii), Clostridium tetani, Corynebacterium, Corynebacterium diphtheriae, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumoniae, Neisseria, Neisseria gonorrhoeae, Neisseria meningitidis, Pasteurella, Pasteurella multocida, Pasteurella tularensis, Peptostreptococcus, Porphyromonas gingivalis, Prevotella melaninogenica (also known as Bacteroides melaninogenicus), Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholerae, Vibrio comma, Vibrio parahaemolyticus, Vibrio vulnificus, Wolbachia, Yersinia, Yersinia enterocolitica, Yersinia pestis, and Yersinia pseudotuberculosis, and other genus and species known to those of skill in the art.

Exemplary genus and species of yeast cells include Saccharomyces, Saccharomyces cerevisiae, Torula, Saccharomyces boulardii, Schizosaccharomyces, Schizosaccharomyces pombe, Candida, Candida glabrata, Candida tropicalis, Yarrowia, Candida parapsilosis, Candida krusei, Saccharomyces pastorianus, Brettanomyces, Brettanomyces bruxellensis, Pichia, Pichia guilliermondii, Cryptococcus, Cryptococcus gattii, Torulaspora, Torulaspora delbrueckii, Zygosaccharomyces, Zygosaccharomyces bailii, Candida lusitaniae, Candida stellata, Geotrichum, Geotrichum candidum, Pichia pastoris, Kluyveromyces, Kluyveromyces marxianus, Candida dubliniensis, Kluyveromyces, Kluyveromyces lactis, Trichosporon, Trichosporon uvarum, Eremothecium, Eremothecium gossypii, Pichia stipitis, Candida milleri, Ogataea, Ogataea polymorpha, Candida oleophilia, Zygosaccharomyces rouxii, Candida albicans, Leucosporidium, Leucosporidium frigidum, Candida viswanathii, Candida blankii, Saccharaomyces telluris, Saccharomyces florentinus, Sporidiobolus, Sporidiobolus salmonicolor, Dekkera, Dekkera anomala, Lachancea, Lachancea kluyveri, Trichosporon, Trichosporon mycotoxinivorans, Rhodotorula, Rhodotorula rubra, Saccharomyces exiguus, Sporobolomyces koalae, and Trichosporon cutaneum, and other genus and species known to those of skill in the art.

Exemplary genus and species of fungal cells include Sac fungi, Basidiomycota, Zygomycota, Chtridiomycota, Basidiomycetes, Hyphomycetes, Glomeromycota, Microsporidia, Blastocladiomycota, and Neocallimastigomycota, and other genus and species known to those of skill in the art.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Production of 3HP

3HP biosensors were co-expressed with the 3HP production pathway in order to monitor 3HP production in real-time. As shown in FIG. 3A, 3HP is produced from glucose by converting malonyl-CoA into malonate semialdehyde and then on to 3HP. Malonyl-CoA reductase (mcr) performs both of these reactions but competes with fatty acid biosynthesis for malonyl-CoA. The production pathway includes the endogenous biosynthesis of malonyl-CoA and the bi-functional enzyme malonyl-CoA reductase (mcr) from the carbon fixation pathway of Chloroflexus aurantiacus (see Mattozzi, M., Ziesack, M., Voges, M. J., Silver, P. A. and Way, J. C. (2013) Expression of the sub-pathways of the Chloroflexus aurantiacus 3-hydroxypropionate carbon fixation bicycle in E. coli: Toward horizontal transfer of autotrophic growth. Metab Eng, 16, 130-139. Mcr shunts malonyl-CoA away from fatty acid biosynthesis by catalyzing the conversion of malonyl-CoA, first into malonate semialdehyde, and then into 3HP at the expense of two NADPH. This route from glucose to 3HP has been published previously, achieving titers of 60 mg/L with expression of mcr alone (see Rathnasingh, C., Raj, S. M., Lee, Y., Catherine, C., Ashok, S. and Park, S. (2012) Production of 3-hydroxypropionic acid via malonyl-CoA pathway using recombinant Escherichia coli strains. Journal of biotechnology, 157, 633-640). Titers were increased to 180 mg/L with overexpression of the ACC complex and pntAB, increasing availability of malonyl-CoA and NADPH, respectively. For our study, we chose to increase the amount of malonyl-CoA available for 3HP production by use of the fatty acid inhibitor cerulenin, rather than through genetic manipulations. Fatty acid biosynthesis is the primary sink for malonyl-CoA and operates at a much higher velocity than heterologously expressed mcr. Since cerulenin inhibits the activities of fabB and fabF, increasing its concentration results in lower fatty acid biosynthesis rates and a higher concentration of available malonyl-CoA (see Heath, R. J. and Rock, C. O. (1995) Regulation of malonyl-CoA metabolism by acyl-acyl carrier protein and beta-ketoacyl-acyl carrier protein synthases in Escherichia coli. The Journal of biological chemistry, 270, 15531-15538. The biosensor helper enzymes, pcs and pcs^{Δ3}/ach, were constitutively expressed while mcr was expressed conditionally with the addition of IPTG.

As shown in FIG. 3B, the acuR-based acrylate (and 3HP) biosensor reports the progress of 3HP production in real-time. The addition of 50 mM glucose (purple line) results in a small increase in fluorescence over background (grey line). Addition of IPTG increases the production of mcr and the activation of the biosensor (blue line). Providing glucose, IPTG and cerulenin together results in the most rapid biosensor activation (green line). As shown in FIG. 3C, fluorescence of the acuR-based biosensor after 12 hours reveals an approximately 5-fold increase in fluorescence in the presence of glucose, IPTG, and cerulenin when compared to 3HP production with glucose alone. In the absence of mcr, IPTG and cerulenin have no effect on biosensor activation (grey bars). Error bars and confidence bands represent the 95% confidence interval (n=3).

### Example II

### Production of Acrylate

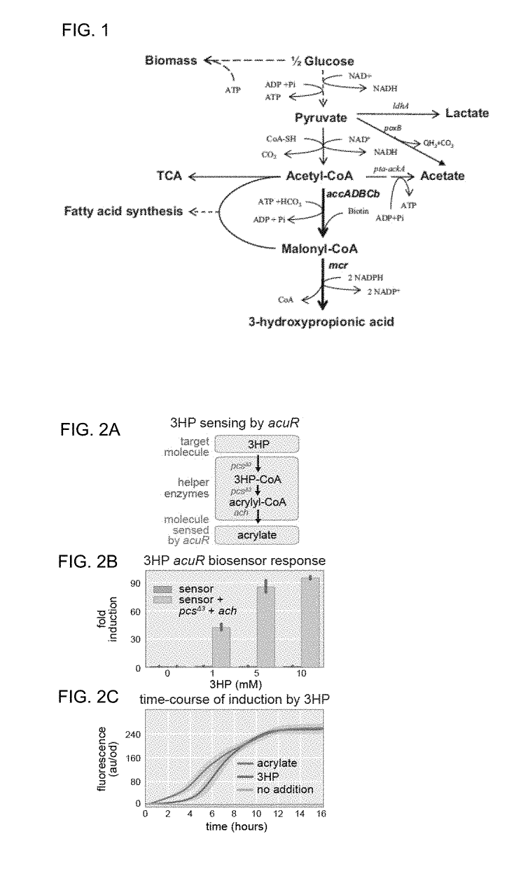

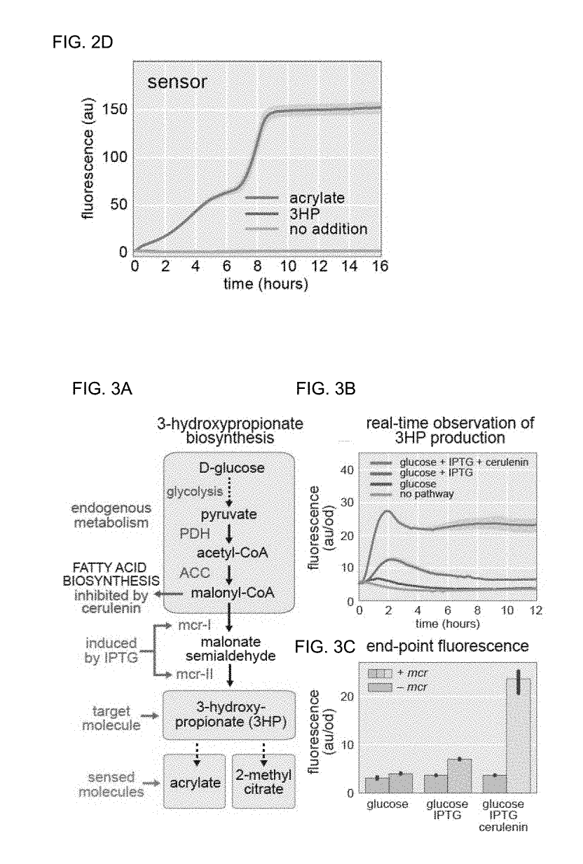

As shown in FIG. 2A, a pathway was constructed that converts 3HP to acrylate. An acrylate biosensor senses the production of acrylate and, therefore, the production of 3HP. The transcriptional regulator acuR is used to regulate transcription of a target gene in proportion to the amount of acrylate present in the cell. Accordingly, the acrylate biosensor reports intracellular 3HP concentration.

The disclosure provides that a truncated version of the multifunctional enzyme pcs (truncPCS) is used to convert 3HP into acrylyl-CoA, which is subsequently hydrolyzed to acrylate by the acrylyl-CoA hydrolase (ach) from Acinetobacter baylyi (see Valle, F., Agard, N.J. and Noriega, C. (2013) Direct biocatalytic production of acrylic acid and other carboxylic acid compounds, PCT/US2012/056639 hereby incorporated by reference in its entirety. In Chloroflexus aurantiacus, pcs catalyzes three subsequent reactions: 3HP to 3HP-CoA to acrylyl-CoA to propionyl-CoA (see Mattozzi, M., Ziesack, M., Voges, M. J., Silver, P. A. and Way, J. C. (2013) Expression of the sub-pathways of the Chloroflexus aurantiacus 3-hydroxypropionate carbon fixation bicycle in E. coli: Toward horizontal transfer of autotrophic growth. Metab Eng, 16, 130-139).

Acrylate production was identified by a fluorescent reporter. AcuR is an allosteric transcriptional regulator that binds acrylate in order to regulate dimethylsulfoniopropionate (DMSP) catabolism in Rhodobacter sphaeroides (see Sullivan, M. J., Curson, A. R., Shearer, N., Todd, J. D., Green, R. T. and Johnston, A. W. (2011) Unusual regulation of a leaderless operon involved in the catabolism of dimethylsulfoniopropionate in Rhodobacter sphaeroides. PloS one, 6, e15972). AcuR is a member of the TetR transcriptional repressor family.

A biosensor was constructed as a single plasmid encoding both the allosteric transcriptional regulator AcuR and a fluorescent reporter. The reporter mRNA is transcribed from a promoter/operator sequence controlled by the allosteric transcriptional regulator AcuR. A medium-strength constitutive promoter (see Davis, J. H., Rubin, A. J. and Sauer, R. T. (2011) Design, construction and characterization of a set of insulated bacterial promoters. Nucleic acids research, 39, 1131-1141) was used to drive regulator transcription. The biosensor was constructed in commonly used high and low copy plasmids to evaluate their behavior in different contexts. High copy plasmids employed a pUC origin of replication (˜100-500 copies), while the low copy plasmids encoded the SC101 replication origin (2-5 copies). All plasmids expressed beta-lactamase, enabling the use of carbenicillin for plasmid maintenance.

For the acuR-based biosensor, accumulation of acrylyl-CoA is necessary. Separation of pcs into its functional domains has been shown to increase the rates of the individual reactions (see Alber, B. E. and Fuchs, G. (2002) Propionyl-coenzyme A synthase from Chloroflexus aurantiacus, a key enzyme of the 3-hydroxypropionate cycle for autotrophic CO2 fixation. The Journal of biological chemistry, 277, 12137-12143 hereby incorporated by reference in its entirety). According to methods described herein, the domain responsible for conversion of acrylyl-CoA to propionyl-CoA was removed from pcs while preserving the activity of the other two domains. The truncated enzyme is referred to herein as pcs^{Δ3}, and its co-expression with ach and acuR constitute the acuR-based acrylate (and 3HP) biosensor (see FIG. 2A). Exogenously supplied 3HP triggers a fluorescent response in cells containing the biosensor (blue bars). Increasing concentrations of 3HP in the media resulted in increasing levels of fluorescence when pcs^{Δ3} and ach were present, but resulted in no biosensor activation in their absence (grey bars) (see FIG. 2B). A90-fold increase in fluorescence was obtained when the acuR-based biosensor was induced with 10 mM 3HP. The induction kinetics of 3HP and the authentic activator acrylate were compared by monitoring biosensor activation in real-time. The fluorescent response of the biosensor to 5 mM 3HP begins immediately and achieves 90% saturation after 8 hours (blue line). (see FIG. 2C). 3HP-mediated induction only slightly lagged the time-course of acrylate induction. Induction by 5 mM acrylate is initially more rapid but achieves the same final fluorescence (green line). Basal induction is low over the duration of the experiment (grey line). Error bars and confidence bands represent the 95% confidence interval (n=3). Fluorescence remained at background levels for greater than 16 hours in the absence of pcs^{Δ3} and ach (FIG. 2D).

Co-expression of pcs^{Δ3} and ach enables in vivo production of acrylate which was determined by LC/MS. See FIG. 5B. While biologically-derived 3HP is used to produce several materials, it is most importantly a precursor to bio-based acrylate. Currently, 3HP is isolated from cell culture and chemically converted to acrylate. In vivo production of acrylate obviates this step by allowing its direct fermentation. 1.62±0.05 mM and 0.27±0.04 mM acrylate was produced from 50 mM and 5 mM 3HP, respectively. Addition of the mcr plasmid resulted in 60±37 μM acrylate produced from glucose in rich media.

### Example III

### Optimizing Acrylate Production

The acuR-based acrylate biosensor was used to determine optimal concentrations of cerulenin and IPTG for 3HP production. While low levels of cerulenin inhibit fatty acid biosynthesis and make additional malonyl-CoA available for 3HP production, higher concentrations result in poor cell viability and reduced 3HP production. To determine the concentration of cerulenin that achieves the correct balance of these opposing effects, eight cerulenin concentrations between 0 and 100 μg/mL were evaluated. Similarly, high concentrations of IPTG result in high mcr gene expression. To determine whether maximal or more nuanced mcr expression would achieve optimal 3HP production, four levels of IPTG induction were evaluated. IPTG and cerulenin concentrations have interacting effects and a combinatorial evaluation of their concentrations was carried out. The acuR-based 3HP biosensor was used to determine cerulenin and IPTG concentrations that result in the highest fluorescence response (20 μg/ml cerulenin and 1000 μM IPTG). Mass spectrometry was used to determined titers for conditions near this point in order to verify that the biosensor was identifying optimal production conditions.

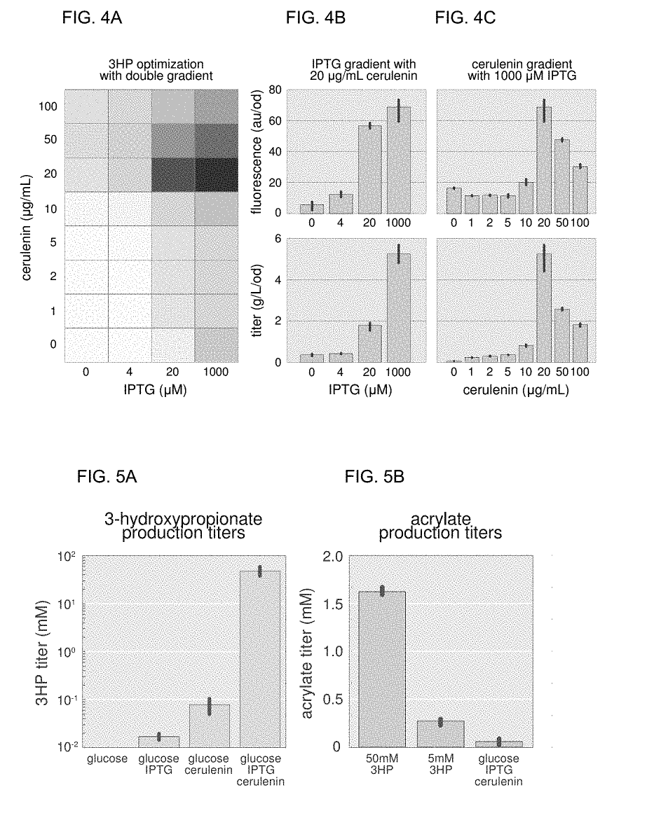

As shown in FIG. 4A, eight concentrations of cerulenin and four concentrations of IPTG were combinatorially evaluated in triplicate. Growth-normalized fluorescence of the 96 samples was measured after 24 hours (darker shades indicate higher fluorescence). 20 μg/mL cerulenin combined with 1000 μM IPTG resulted in the greatest biosensor response. As shown in FIG. 4B, fluorescence resulting from the acrylate sensor was determined for a range of IPTG concentrations while cerulenin was held constant at 20 μg/mL. As shown in FIG. 4C, fluorescence resulting from the acrylate sensor was determined for a range of cerulenin concentrations while IPTG was held constant at 1000 μM. In both cases, fluorescence indicated the optimal concentrations of IPTG and cerulenin. Error bars represent the 95% confidence interval (n=3).

### Example IV

### Recovering Acrylate from Bacteria Cells

Recovery of acrylate from bacteria cells is contemplated as part of the present disclosure. Acrylate may be recovered from the genetically modified bacteria cells described herein by methods known to those of skill in the art. Such methods include separation or purification techniques known to separate or purify acrylic acid from other constituents in a fluid. The disclosure contemplates methods of continually removing acrylate from a cell. The disclosure contemplates methods of continually removing acrylate from a cell, so as to maintain the viability of the cell, such as when acrylate within a cell may lead to cell death.

### Example V

### Chemicals and Reagents

### Chemicals and Reagents

All reagents were obtained from Sigma unless otherwise noted. Antibiotics and IPTG were obtained from Gold Biotechnology. PCR mix was purchased from Kapa Biosystems. 3-hydroxypropionate was purchased from Toronto Research Chemicals. Cerulenin was purchased from Cayman Chemical and dissolved in ethanol. Acrylic acid was stored at room temperature with 200 ppm MEHQ as an inhibitor and diluted immediately prior to use. All cell culture additives were dissolved in deionized water to achieve appropriate working concentrations.

### Strains and Plasmids

Plasmids were constructed using Gibson isothermal assembly methods (see Gibson, D. G., Young, L., Chuang, R. Y., Venter, J. C., Hutchison, C. A., 3rd and Smith, H. O. (2009) Enzymatic assembly of DNA molecules up to several hundred kilobases. Nat Methods, 6, 343-345) and cloned into DH5α electrocompetent cells purchased from New England Biolabs. Biosynthesis of product molecules was carried out in either BL21 (DE3) or DH5α. The acuR-based acrylate biosensor is composed of two plasmids. The first is the previously characterized high-copy acrylate biosensor pJKR-H-acuR (see Rogers, J. K., Guzman, C. D., Taylor, N. D., Raman, S., Anderson, K. and Church, G. M. (2015) Synthetic biosensors for precise gene control and real-time monitoring of metabolites. Nucleic acids research, 43, 7648-7660 hereby incorporated by reference in its entirety (Addgene plasmid #62567)), which expresses sfGFP under the control of the acrylate responsive transcription factor, acuR, on a pUC origin of replication providing (3-lactam resistance. The second is derived from pJKR-PCS such that PCS is truncated between amino acids 1400 and 1401. The enzyme acrylyl-CoA hydrolase from Acinetobacter baylyi was subsequently cloned into the plasmid under the control of the P2 constitutive promoter (see Mutalik, V. K., Guimaraes, J. C., Cambray, G., Lam, C., Christoffersen, M. J., Mai, Q. A., Tran, A. B., Paull, M., Keasling, J. D., Arkin, A. P. et al. (2013) Precise and reliable gene expression via standard transcription and translation initiation elements. Nat Methods, 10, 354-360). The resulting plasmid is designated pJKR-PCSfrag-ACH. The 3HP biosynthesis plasmid, designated pJKR-MCR, was constructed such that malonyl-CoA reductase from Chloroflexus aurantiacus was expressed by the pLlacO promoter (Lutz, R. and Bujard, H. (1997) Independent and tight regulation of transcriptional units in Escherichia coli via the LacR/O, the TetR/O and AraC/I1-I2 regulatory elements. Nucleic acids research, 25, 1203-1210) under the control of Lad on a p15a origin of replication with spectinomycin resistance.

### 3-Hydroxypropionate Biosensor Characterization

DH5α cells doubly transformed with plasmids pPro24-GFP and pJKR-PCS, or plasmids pJKR-H-acuR and pJKR-PCSfrag-ACH, were exposed to increasing concentrations of 3HP and monitored for GFP expression. Cells were grown overnight to saturation before being diluted 1:100 into fresh LB media and incubated at 200 RPM and 37° C. After four hours, 150 μl of the log-phase cells were transferred to 96-well plates and 3HP was added to the appropriate final concentration. Each inoculation and induction was performed in triplicate. Strains lacking the biosensor helper plasmids were included to demonstrate that the biosensor activation was dependent on the presence of the helper plasmids. In the case of the end-point measurements, fluorescence was measured 16 hours after 3HP addition with a Biotek HT plate reader (excitation 485/20, emission 528/20). Time course data was collected over a 16 hour period on the same plate reader at 37° C. with fast shaking and 10 minute measurement intervals. Fluorescence was normalized by optical density. Fold induction was determined by dividing the fluorescence obtained in the presence of the inducer molecule by the level of fluorescence observed in the absence of any inducer. Error bars represent the 95% confidence interval derived from the standard error of the mean. In all cases the standard error was determined by resampling with replacement (n=5000).

### Optimization of 3-Hydroxypropionate Production Conditions

DH5α cells transformed with plasmids pJKR-H-acuR, pJKR-PCSfrag-ACH and pJKR-MCR were grown to saturation overnight at 37° C. in selective LB media. The cells were diluted 1:100 in selective LB media and cultured for four hours prior to being transferred to 96-well deep well blocks (working volume 1 mL). IPTG and cerulenin were added such that each combination of concentrations for IPTG and cerulenin were evaluated in triplicate. The cerulenin concentrations evaluated were 0, 1, 2, 5, 10, 20, 50 and 100 μg/mL. The IPTG concentrations evaluated were 0, 4, 20 and 1000 μM. The deep well block was incubated humidified at 37° C. and 900 RPM for 24 hours. 150 μl was then transferred to a microtiter plate and fluorescence and optical density were measured in the Biotek HT plate reader as previously described. 300 μl of the cell suspension was prepared for analysis by LC/MS.

### 3-Hydroxypropionate/Acrylate Production and Monitoring

DH5α cells containing the plasmid for the acuR-based acrylate (and 3HP) biosensor was transformed with the plasmid pJKR-MCR. The production strain was grown up overnight and back-diluted 1:100 into fresh LB media and incubated at 200 RPM and 37° C. After four hours, 150 μl of the log-phase cells were transferred to 96-well plates and exposed to 3HP production conditions. The acuR-based biosensor production strain was incubated with 50 mM glucose and different combinations of 1 mM IPTG and 20 μg/ml cerulenin. Growth-normalized fluorescence was observed in the Biotek HT plate reader. End-point measurements were taken after 12 hours. 3HP production titers were determined by liquid-chromatography and mass spectrometry (LC/MS). The strain used for 3HP titer measurements only contained the production plasmid pJKR-MCR. Overnight cultures were inoculated 1:100 into 1 mL of LB supplemented with 1 mM IPTG, 20 μg/ml cerulenin and 50 mM glucose in 96-well blocks. Production took place at 900 RPM and 37 C for 16 hours before supernatants were isolated and filtered at 0.2 μm for LC/MS. Samples were prepared for acrylate analysis in an identical manner with DH5α as the strain background. Production of acrylate from glucose was carried out with 50 mM glucose, 1 mM IPTG and 20 μg/ml cerulenin. Acrylate was measured on a Thermo q-Exactive mass spectrometer equipped with a Thermo 3000 Ultimate μHPLC. A resolution of 70,000 was used on the mass spectrometer. A hydrophilic interaction chromatography method was used using an EMD Sequant pHILIC column (150 mm length, 2.1 mm ID, 5 μm particle size) at a flow rate of 100 μL/minute. Mobile phase A was 20 mM ammonium carbonate and B was acetonitrile. The gradient started at 100% B and linearly decreased to 40% B over 20 minutes. B was then decreased further to 20% over 10 minutes, and then returned to initial conditions at 100% B over 0.1 minutes and maintained for the next 11.9 minutes to equilibrate the column for the following run. All production runs were setup in triplicate. Error bars represent the 95% confidence interval derived from the standard error of the mean.

### Sequences

TABLE 1
Sequence of regulator protein and cognate promoter/operator.

| | Promoter / Operator | |
|---|---|---|
| Regulator | Sequence | Regulator Sequence |
| acuR | GCTTCACAACCGCACTTGA | ATGCCGCTGACCGACACCCCGCCGTCTGTTCCGCAGAAACCGCGTCGTGGTCGTCCGCGTGGTGCTCCG |
| | TTTAATAGACCATACCGTC | GACGCTTCTCTGGCTCACCAGTCTCTGATCCGTGCTGGTCTGGAACACCTGACCGAAAAAGGTTACTCT |
| | TATTATTTCTGG | TCTGTTGGTGTTGACGAAATCCTGAAAGCTGCTCGTGTTCCGAAAGGTTCTTTCTACCACTACTTCCGT |
| | | AACAAAGCTGACTTCGGTCTGGCTCTGATCGAAGCTTACGACACCTACTTCGCTCGTCTCCTCGACCAG |
| | | GCGTTCCTGGACGGTTCGCTGGCTCCGCTGGCTCGTCTGCGTCTGTTCACCCGTATGGCTGAAGAAGGT |
| | | ATGGCTCGTCACGGTTTCCGTCGTGGTTGCCTGGTTGGTAACCTGGGTCAGGAAATGGGTGCTCTGCCG |
| | | GACGACTTCCGTGCTGCTCTGATCGGTGTTCTGGAAACCTGGCAGCGTCGTACCGCTCAGCTGTTCCGT |
| | | GAAGCTCAGGCTTGCGGTGAACTGTCTGCTGACCACGACCCGGACGCTCTGGCTGAAGCTTTCTGGATC |
| | | GGTTGGGAAGGTGCTATCCTGCGTGCTAAACTGGAACTGCGTCCGGACCCGCTGCACTCTTTCACCCGT |
| | | ACCTTCGGTCGTCACTTCGTTACCCGTACCCAGGAATAA |

The following sequence for the truncated PCS is provided as follows:

ATGATCGATACCGCACCGCTGGCACCGCCGCGTGCTCCGCGCAGCAATCC
GATTCGTTCGCGTGGATTGGGAAGCGCAGC
GTGCAGCAGCACTGGCCGATCCGGGTGCATTTCATGGTGCGATCGCCCGT
ACCGTTATTCACTGGTATGATCCGCAGCAT
CACTGCTGGATTCGCTTCAACGAAAGCTCTCAGCGTTGGGAAGGTCTGGA
TGCAGCAACGGGTGCTCCGGTTACAGTGGA
TTATCCTGCCGATTACCAGCCGTGGCAGCAGGCATTTGATGATAGTGAAG
CGCCGTTTTATCGCTGGTTCAGCGGCGGTC
TGACGAACGCATGTTTTAATGAAGTTGATCGTCACGTGACAATGGGTTAC
GGCGATGAAGTGGCGTATTACTTCGAAGGT
GATCGCTGGGATAATAGCCTGAACAATGGCCGTGGCGGTCCGGTGGTTCA
GGAAACGATTACCCGTCGCCGTCTGCTGGT
TGAAGTGGTTAAAGCAGCGCAGGTTCTGCGCGATCTGGGCCTGAAAAAAG
GTGATCGTATCGCGCTGAACATGCCGAATA
TCATGCCGCAGATTTATTACACCGAAGCCGCAAAACGCCTGGGTATTCTG
TATACGCCGGTGTTTGGCGGTTTCAGTGAT
AAAACCCTGAGCGATCGCATCCATAATGCAGGTGCGCGTGTGGTTATTAC
CTCTGATGGCGCGTATCGTAACGCCCAGGT
GGTTCCGTATAAAGAAGCCTACACGGATCAGGCACTGGATAAATACATCC
CGGTGGAAACCGCCCAGGCAATTGTTGCAC
AGACGCTGGCAACCCTGCCGCTGACCGAAAGTCAGCGCCAGACGATTATC
ACCGAAGTGGAAGCAGCACTGGCAGGTGAA
ATTACGGTTGAACGTTCTGATGTTATGCGCGGTGTGGGCAGTGCGCTGGC
CAAACTGCGCGATCTGGATGCCAGTGTGCA
GGCAAAAGTTCGTACCGTGCTGGCACAGGCGCTGGTTGAAAGCCCGCCGC
GCGTGGAAGCAGTGGTTGTGGTTCGTCATA
CGGGTCAGGAAATCCTGTGGAATGAAGGCCGTGATCGCTGGAGCCACGAT
CTGCTGGATGCAGCACTGGCGAAAATTCTG
GCTAACGCACGCGCCGCAGGTTTTGATGTTCACTCTGAAAACGATCTGCT
GAATCTGCCGGATGATCAGCTGATCCGTGC
TCTGTATGCGAGTATTCCGTGCGAACCAGTTGATGCCGAATATCCGATGT
TTATTATCTACACGAGCGGTTCTACCGGCA
AACCGAAAGGTGTTATTCATGTTCACGGCGGTTACGTGGCGGGCGTGGTT
CATACCCTGCGCGTTAGTTTCGATGCCGAA
CCGGGCGATACGATTTATGTGATCGCAGATCCGGGCTGGATCACAGGTCA
GAGCTACATGCTGACGGCAACCATGGCAGG
TCGTCTGACTGGTGTGATTGCCGAAGGTTCTCCGCTGTTTCCGAGTGCGG
GCCGCTATGCCTCTATTATCGAACGTTACG
GTGTTCAGATTTTTAAAGCGGGCGTTACGTTCCTGAAAACCGTGATGAGT
AACCCGCAGAATGTTGAAGATGTGCGCCTG
TATGATATGCACAGTCTGCGTGTGGCAACCTTTTGTGCAGAGCCGGTTAG
CCCGGCAGTGCAGCAGTTCGGTATGCAGAT
CATGACGCCGCAGTATATTAATAGCTACTGGGCGACGGAACATGGCGGTA
TTGTGTGGACCCACTTTTATGGCAACCAGG
ATTTCCCGCTGCGTCCAGATGCACATACGTACCCGCTGCCGTGGGTTATG
GGTGATGTTTGGGTGGCAGAAACCGATGAA
TCTGGCACCACGCGCTATCGCGTGGCGGATTTCGATGAAAAAGGTGAAAT
CGTTATCACCGCACCGTATCCGTACCTGAC
GCGAACCCTGTGGGGTGATGTGCCGGGTTTTGAAGCGTATCTGCGTGGTG
AAATCCCGCTGCGTGCATGGAAAGGTGATG
CAGAACGTTTCGTTAAAACCTACTGGCGTCGTGGTCCGAATGGCGAATGG
GGTTATATCCAGGGCGATTTTGCGATTAAA
TACCCGGATGGTAGTTTCACGCTGCATGGCCGCAGCGATGATGTTATTAA
TGTGTCCGGCCACCGTATGGGTACGGAAGA
AATCGAAGGTGCCATTCTGCGTGATCGCCAGATCACCCCGGATTCTCCGG
TGGGTAACTGCATTGTGGTTGGCGCGCCGC
ATCGTGAAAAAGGCCTGACCCCGGTTGCATTTATCCAGCCAGCACCGGGT
CGTCACCTGACGGGTGCAGATCGCCGTCGC
CTGGATGAACTGGTGCGTACCGAAAAAGGTGCAGTTAGCGTGCCGGAAGA
TTATATTGAAGTTAGTGCGTTTCCGGAAAC
CCGCAGCGGTAAATACATGCGTCGCTTCCTGCGTAATATGATGCTGGATG
AACCGCTGGGCGATACCACGACCCTGCGCA
ACCCGGAAGTGCTGGAAGAAATCGCGGCCAAAATTGCCGAATGGAAACGT
CGCCAGCGCATGGCAGAAGAACAGCAGATT
ATCGAACGTTATCGCTACTTTCGTATTGAATATCATCCGCCGACCGCAAG
TGCAGGTAAACTGGCAGTGGTTACGGTTAC
CAATCCGCCGGTGAACGCCCTGAATGAACGTGCTCTGGATGAACTGAACA
CCATCGTGGATCACCTGGCGCGTCGCCAGG
ATGTTGCAGCGATTGTGTTTACGGGTCAGGGTGCTCGCAGCTTCGTGGCC
GGTGCGGATATCCGTCAGCTGCTGGAAGAA
ATTCATACCGTTGAAGAAGCCATGGCACTGCCGAACAATGCGCACCTGGC
CTTTCGCAAAATTGAACGTATGAACAAACC
GTGCATTGCCGCAATCAATGGTGTGGCACTGGGCGGTGGCCTGGAATTTG
CGATGGCCTGTCATTATCGCGTTGCCGATG
TGTACGCAGAATTTGGTCAGCCGGAAATCAACCTGCGTCTGCTGCCGGGT
TATGGTGGTACGCAGCGTCTGCCGCGTCTG
CTGTACAAACGCAACAATGGTACAGGCCTGCTGCGTGCGCTGGAAATGAT
TCTGGGTGGCCGCAGCGTGCCAGCAGATGA
AGCACTGGAACTGGGTCTGATTGATGCAATCGCGACCGGCGATCAGGATA
GTCTGAGCCTGGCCTGCGCACTGGCGCGTG
CGGCAATCGGTGCAGATGGTCAGCTGATTGAAAGCGCAGCGGTGACCCAG
GCCTTTCGTCATCGCCACGAACAGCTGGAT
GAATGGCGTAAACCGGACCCGCGCTTCGCGGATGATGAACTGCGCTCTAT
TATCGCCCATCCGCGTATCGAACGCATTAT
CCGTCAGGCGCATACCGTTGGTCGTGATGCAGCAGTGCACCGTGCACTGG
ATGCAATTCGTTATGGCATTATCCATGGTT
TTGAAGCCGGCCTGGAACACGAAGCAAAACTGTTCGCCGAAGCAGTGGTT
GATCCGAATGGTGGCAAACGCGGCATCCGT
GAATTTCTGGATCGTCAGTCTGCACCGCTGCCGACACGTCGCCCGCTGAT
TACCCCGGAACAGGAACAGCTGCTGCGTGA
TCAGAAAGAACTGCTGCCGGTGGGTAGTCCGTTTTTCCCTGGCGTTGATC
GCATCCCGAAATGGCAGTATGCGCAGGCCG
TGATTCGTGATCCCGATACTGGTGCAGCAGCACATGGCGATCCGATCGTT
GCGGAAAAACAGATTATCGTTCCGGTGGAA
CGTCCGCGTGCGAACCAGGCACTGATTTACGTTCTGGCGAGCGAAGTGAA
CTTTAATGATATTTGGGCCATCACAGGTAT
TCCGGTGAGCCGCTTCGATGAACATGATCGTGATTGGCACGTGACGGGTT
CTGGTGGCATCGGCCTGATTGTTGCGCTGG
GCGAAGAAGCCCGTCGCGAAGGTCGTCTGAAAGTTGGCGATCTGGTGGCG
ATCTATAGCGGCCAGTCTGATCTGCTGAGC
CCGCTGATGGGTCTGGACCCGATGGCAGCCGATTTTGTGATTCAGGGTAA
TGATACCCCGGATGGCTCTCATCAGCAGTT
CATGCTGGCACAGGCACCGCAGTGCCTGCCGATCCCGACGGATATGAGCA
TTGAAGCAGCGGGTTCTTATATCCTGAACC
TGGGCACCATTTACCGCGCACTGTTTACGACCCTGCAATAA

The following sequence for ACH is provided as follows:

ATGCTGGATGCACATATCAGCCCCGAGGGCACGTTAAGTTTGCAGACCAT
CGCCATGCCGGCTGATACGAATTGGTCTGG
CGATGTTTTTGGTGGTTGGATTGTGTCTCAGATGGATCTGGCGGGTGCGA
TTCATGCCGAGCGTTTCTCTAAGGGCCGCT
GTGCGACGATTTCTATTAACCAGATGACCTTTTTAGTTCCTGTGAAAGTT
GGTGACGTGATTTCGTGCTACACCAAAATT
TTAAAAGTCGGAAACACTTCGATTCAGATGCAGATCGAAGTCTGGGATAG
CCACGATTCAAGCCGGCCGCCGAAACGGGT
AACCGAAGGGGTGTTTACATTTGTCGCCGTGGACGTTAAAGGCAATAAAC
GCACCATCGCCGAAGACTTAAAACAACAGT
TCCTGCAGCACGCGAGCTAA

1. A method of producing acrylate in vivo in a genetically modified bacteria cell comprising
providing a cell with a first one or more nucleic acids encoding a pathway resulting in the production of 3-hydroxypropionate,
providing the cell with a second one or more nucleic acids encoding a truncated pcs enzyme lacking a domain converting acrylyl-CoA to propionyl-CoA,
providing the cell with a third one or more nucleic acids encoding acrylyl-CoA hydrolase,
wherein the cell expresses the first, second and third one or more nucleic acids and produces acrylate.

2. The method of claim 1 wherein the cell produces 3HP which is converted by the truncated pcs enzyme to acrylyl-CoA which is converted to acrylate by the acrylyl-CoA hydrolase.

3. The method of claim 1 wherein the acrylate is recovered from the cell.

4. The method of claim 1 wherein fatty acid biosynthesis within the cell is inhibited.

5. The method of claim 1 wherein fatty acid biosynthesis within the cell is inhibited by exposing the cell to a fatty acid inhibitor.

6. The method of claim 1 wherein fatty acid biosynthesis within the cell is inhibited by exposing the cell to cerulenin.

7. The method of claim 1 wherein fatty acid biosynthesis within the cell is inhibited thereby increasing malonyl-CoA for processing by malonyl-CoA reductase.

8. The method of claim 1 wherein the genetically modified bacteria cell is grown into a population of genetically modified bacteria cells and the acrylate is recovered.

9. A method of genetically modifying a bacteria cell comprising
providing a cell with a first one or more foreign or exogenous nucleic acids encoding a pathway resulting in the production of 3-hydroxypropionate,
providing the cell with a second one or more foreign or exogenous nucleic acids encoding a truncated pcs enzyme lacking a domain converting acrylyl-CoA to propionyl-CoA, and
providing the cell with a third one or more foreign or exogenous nucleic acids encoding acrylyl-CoA hydrolase.

10. A genetically modifying a bacteria cell comprising
a first one or more foreign or exogenous nucleic acids encoding a pathway resulting in the production of 3-hydroxypropionate,
a second one or more foreign or exogenous nucleic acids encoding a truncated pcs enzyme lacking a domain converting acrylyl-CoA to propionyl-CoA, and
a third one or more foreign or exogenous nucleic acids encoding acrylyl-CoA hydrolase.


SEQUENCE LISTING (4 sequences)

SEQ ID NO: 1 - DNA, 50 bp, Artificial Sequence
  Description of Artificial Sequence Synthetic oligonucleotide
gcttcacaaccgcacttgatttaatagaccataccgtctattatttctgg

SEQ ID NO: 2 - DNA, 660 bp, Artificial Sequence
  Description of Artificial Sequence Synthetic polynucleotide
atgccgctgaccgacaccccgccgtctgttccgcagaaaccgcgtcgtggtcgtccgcgtggtgctccggacgcttctctggctcaccagtctctgatccgtgctggtctggaacacctgaccgaaaaaggttactcttctgttggtgttgacgaaatcctgaaagctgctcgtgttccgaaaggttctttctaccactacttccgtaacaaagctgacttcggtctggctctgatcgaagcttacgacacctacttcgctcgtctcctcgaccaggcgttcctggacggttcgctggctccgctggctcgtctgcgtctgttcacccgtatggctgaagaaggtatggctcgtcacggtttccgtcgtggttgcctggttggtaacctgggtcaggaaatgggtgctctgccggacgacttccgtgctgctctgatcggtgttctggaaacctggcagcgtcgtaccgctcagctgttccgtgaagctcaggcttgcggtgaactgtctgctgaccacgacccggacgctctggctgaagctttctggatcggttgggaaggtgctatcctgcgtgctaaactggaactgcgtccggacccgctgcactctttcacccgtaccttcggtcgtcacttcgttacccgtacccaggaataa

SEQ ID NO: 3 - DNA, 4201 bp, Artificial Sequence
  Description of Artificial Sequence Synthetic polynucleotide
atgatcgataccgcaccgctggcaccgccgcgtgctccgcgcagcaatccgattcgttcgcgtggattgggaagcgcagcgtgcagcagcactggccgatccgggtgcatttcatggtgcgatcgcccgtaccgttattcactggtatgatccgcagcatcactgctggattcgcttcaacgaaagctctcagcgttgggaaggtctggatgcagcaacgggtgctccggttacagtggattatcctgccgattaccagccgtggcagcaggcatttgatgatagtgaagcgccgttttatcgctggttcagcggcggtctgacgaacgcatgttttaatgaagttgatcgtcacgtgacaatgggttacggcgatgaagtggcgtattacttcgaaggtgatcgctgggataatagcctgaacaatggccgtggcggtccggtggttcaggaaacgattacccgtcgccgtctgctggttgaagtggttaaagcagcgcaggttctgcgcgatctgggcctgaaaaaaggtgatcgtatcgcgctgaacatgccgaatatcatgccgcagatttattacaccgaagccgcaaaacgcctgggtattctgtatacgccggtgtttggcggtttcagtgataaaaccctgagcgatcgcatccataatgcaggtgcgcgtgtggttattacctctgatggcgcgtatcgtaacgcccaggtggttccgtataaagaagcctacacggatcaggcactggataaatacatcccggtggaaaccgcccaggcaattgttgcacagacgctggcaaccctgccgctgaccgaaagtcagcgccagacgattatcaccgaagtggaagcagcactggcaggtgaaattacggttgaacgttctgatgttatgcgcggtgtgggcagtgcgctggccaaactgcgcgatctggatgccagtgtgcaggcaaaagttcgtaccgtgctggcacaggcgctggttgaaagcccgccgcgcgtggaagcagtggttgtggttcgtcatacgggtcaggaaatcctgtggaatgaaggccgtgatcgctggagccacgatctgctggatgcagcactggcgaaaattctggctaacgcacgcgccgcaggttttgatgttcactctgaaaacgatctgctgaatctgccggatgatcagctgatccgtgctctgtatgcgagtattccgtgcgaaccagttgatgccgaatatccgatgtttattatctacacgagcggttctaccggcaaaccgaaaggtgttattcatgttcacggcggttacgtggcgggcgtggttcataccctgcgcgttagtttcgatgccgaaccgggcgatacgatttatgtgatcgcagatccgggctggatcacaggtcagagctacatgctgacggcaaccatggcaggtcgtctgactggtgtgattgccgaaggttctccgctgtttccgagtgcgggccgctatgcctctattatcgaacgttacggtgttcagatttttaaagcgggcgttacgttcctgaaaaccgtgatgagtaacccgcagaatgttgaagatgtgcgcctgtatgatatgcacagtctgcgtgtggcaaccttttgtgcagagccggttagcccggcagtgcagcagttcggtatgcagatcatgacgccgcagtatattaatagctactgggcgacggaacatggcggtattgtgtggacccacttttatggcaaccaggatttcccgctgcgtccagatgcacatacgtacccgctgccgtgggttatgggtgatgtttgggtggcagaaaccgatgaatctggcaccacgcgctatcgcgtggcggatttcgatgaaaaaggtgaaatcgttatcaccgcaccgtatccgtacctgacgcgaaccctgtggggtgatgtgccgggttttgaagcgtatctgcgtggtgaaatcccgctgcgtgcatggaaaggtgatgcagaacgtttcgttaaaacctactggcgtcgtggtccgaatggcgaatggggttatatccagggcgattttgcgattaaatacccggatggtagtttcacgctgcatggccgcagcgatgatgttattaatgtgtccggccaccgtatgggtacggaagaaatcgaaggtgccattctgcgtgatcgccagatcaccccggattctccggtgggtaactgcattgtggttggcgcgccgcatcgtgaaaaaggcctgaccccggttgcatttatccagccagcaccgggtcgtcacctgacgggtgcagatcgccgtcgcctggatgaactggtgcgtaccgaaaaaggtgcagttagcgtgccggaagattatattgaagttagtgcgtttccggaaacccgcagcggtaaatacatgcgtcgcttcctgcgtaatatgatgctggatgaaccgctgggcgataccacgaccctgcgcaacccggaagtgctggaagaaatcgcggccaaaattgccgaatggaaacgtcgccagcgcatggcagaagaacagcagattatcgaacgttatcgctactttcgtattgaatatcatccgccgaccgcaagtgcaggtaaactggcagtggttacggttaccaatccgccggtgaacgccctgaatgaacgtgctctggatgaactgaacaccatcgtggatcacctggcgcgtcgccaggatgttgcagcgattgtgtttacgggtcagggtgctcgcagcttcgtggccggtgcggatatccgtcagctgctggaagaaattcataccgttgaagaagccatggcactgccgaacaatgcgcacctggcctttcgcaaaattgaacgtatgaacaaaccgtgcattgccgcaatcaatggtgtggcactgggcggtggcctggaatttgcgatggcctgtcattatcgcgttgccgatgtgtacgcagaatttggtcagccggaaatcaacctgcgtctgctgccgggttatggtggtacgcagcgtctgccgcgtctgctgtacaaacgcaacaatggtacaggcctgctgcgtgcgctggaaatgattctgggtggccgcagcgtgccagcagatgaagcactggaactgggtctgattgatgcaatcgcgaccggcgatcaggatagtctgagcctggcctgcgcactggcgcgtgcggcaatcggtgcagatggtcagctgattgaaagcgcagcggtgacccaggcctttcgtcatcgccacgaacagctggatgaatggcgtaaaccggacccgcgcttcgcggatgatgaactgcgctctattatcgcccatccgcgtatcgaacgcattatccgtcaggcgcataccgttggtcgtgatgcagcagtgcaccgtgcactggatgcaattcgttatggcattatccatggttttgaagccggcctggaacacgaagcaaaactgttcgccgaagcagtggttgatccgaatggtggcaaacgcggcatccgtgaatttctggatcgtcagtctgcaccgctgccgacacgtcgcccgctgattaccccggaacaggaacagctgctgcgtgatcagaaagaactgctgccggtgggtagtccgtttttccctggcgttgatcgcatcccgaaatggcagtatgcgcaggccgtgattcgtgatcccgatactggtgcagcagcacatggcgatccgatcgttgcggaaaaacagattatcgttccggtggaacgtccgcgtgcgaaccaggcactgatttacgttctggcgagcgaagtgaactttaatgatatttgggccatcacaggtattccggtgagccgcttcgatgaacatgatcgtgattggcacgtgacgggttctggtggcatcggcctgattgttgcgctgggcgaagaagcccgtcgcgaaggtcgtctgaaagttggcgatctggtggcgatctatagcggccagtctgatctgctgagcccgctgatgggtctggacccgatggcagccgattttgtgattcagggtaatgataccccggatggctctcatcagcagttcatgctggcacaggcaccgcagtgcctgccgatcccgacggatatgagcattgaagcagcgggttcttatatcctgaacctgggcaccatttaccgcgcactgtttacgaccctgcaataa

SEQ ID NO: 4 - DNA, 420 bp, Artificial Sequence
  Description of Artificial Sequence Synthetic polynucleotide
atgctggatgcacatatcagccccgagggcacgttaagtttgcagaccatcgccatgccggctgatacgaattggtctggcgatgtttttggtggttggattgtgtctcagatggatctggcgggtgcgattcatgccgagcgtttctctaagggccgctgtgcgacgatttctattaaccagatgacctttttagttcctgtgaaagttggtgacgtgatttcgtgctacaccaaaattttaaaagtcggaaacacttcgattcagatgcagatcgaagtctgggatagccacgattcaagccggccgccgaaacgggtaaccgaaggggtgtttacatttgtcgccgtggacgttaaaggcaataaacgcaccatcgccgaagacttaaaacaacagttcctgcagcacgcgagctaa